(19) United States
(12) Patent Application Publication
(10) Pub. No.: US 2026/0174414 A1
(43) Pub. Date: Jun. 25, 2026

(54) SYSTEM AND METHODS FOR A NAVIGATION SYSTEM FOR FACIAL AESTHETIC PROCEDURES

(71) Applicant: Berman Medical, Inc., San Diego, CA (US)

(72) Inventors: David BERMAN, San Diego, CA (US); Andrew HANNA, San Marcos, CA (US)

(73) Assignee: Berman Medical, Inc., San Diego, CA (US)

(21) Appl. No.: 19/415,518

(22) Filed: Dec. 10, 2025

Related U.S. Application Data
(60) Provisional application No. 63/736,048, filed on Dec. 19, 2024.

(51) Int. Cl. A61B 8/00; A61B 34/10; A61B 34/20

(52) U.S. Cl. CPC A61B 8/483; A61B 8/4227; A61B 8/461; A61B 34/10; A61B 34/20; A61B 2034/105; A61B 2034/107; A61B 2034/2051; A61B 2034/2055

(57) ABSTRACT

Described herein is a system and methods for planning an aesthetic procedure on a patient, the system including: a tracker configured to generate a three-dimensional image of a face of the patient; an ultrasound probe, the ultrasound probe configured to generate an ultrasound image of the face of the patient, wherein the ultrasound image and the three-dimensional image are registered into a patient-specific composite model; and a display, wherein the display shows the patient-specific composite model created by identification of anatomical features of the patient-specific composite model, wherein the display shows a risk map derived from the patient-specific composite model, the risk map including one or more safety regions and one or more risk regions, wherein the patient-specific composite model is configured to be evaluated to select one or more target locations on the face of the patient constrained by the risk map.

### FIELD OF THE INVENTION

This application claims priority to U.S. Provisional Application No. 63/736,048, filed Dec. 19, 2024.

### FIELD OF THE INVENTION

The present invention relates to the field of aesthetic dermatology and cosmetic procedures, specifically to systems that enhance precision and safety during facial injectables and related treatments through augmented reality and ultrasound imaging.

### BACKGROUND OF THE INVENTION

Existing methods for facial aesthetic procedures rely heavily on practitioner experience and manual techniques. Current dermal filler procedures involve a needle or cannula placed by a provider to deliver small amounts of filler into targeted layers of tissue, manually adjusting placement and molding the product to achieve symmetry and contour. However, current workflows lack real-time, patient-specific 3D vascular mapping in with unburdened usability. Existing scanning tools (e.g., ultrasound, infrared, augmented reality filters) lack 3D and depth integration or risk-aware guidance.

Further, after the injections, the patient may experience temporary swelling, redness, or bruising. While most effects are mild and short-lived, complications such as infection or vascular occlusion still occur. For example, 62% of experienced practitioners have experienced at least one severe complication in their practice. In addition, 59% of consumers fear suffering a side effect from the procedure.

Fears of safety risks and unpredictable results keep about 15-20 million patients out of the non-invasive aesthetics market. Regarding safety, vascular occlusions can result in severe complications such as scarring, skin necrosis, and blindness. Regarding outcome variability, patients are often hesitant about the precision and predictability of such procedures. Current approaches lack real-time guidance, 3D modeling, or face tracking and classification, limiting accuracy and increasing the risk of complications such as vascular occlusion.

Accordingly, there is a need for aesthetic systems and methods that can be performed with personalized precision and predictability while preventing complications often common with aesthetic procedures.

### SUMMARY OF THE INVENTION

In some variations, the techniques described herein relate to a system for planning an aesthetic procedure on a patient, the system including: a tracker configured to generate a three-dimensional image of a face of the patient; an ultrasound probe, the ultrasound probe configured to generate an ultrasound image of the face of the patient, wherein the ultrasound image and the three-dimensional image are registered into a patient-specific composite model; and a display, wherein the display shows the patient-specific composite model created by identification of anatomical features of the patient-specific composite model, wherein the display shows a risk map derived from the patient-specific composite model, the risk map including one or more safety regions and one or more risk regions based on spatial proximity of the vasculature of the patient to calculated injection trajectories, wherein the patient-specific composite model is configured to be evaluated to select one or more target locations on the face of the patient constrained by the risk map.

In some variations, the techniques described herein relate to a system, further including a syringe configured to inject the one or more target locations with a filler material, wherein the syringe is guided by analysis of the patient-specific composite model.

In some variations, the techniques described herein relate to a system, wherein the syringe includes a sensor module, the sensor module including a housing configured to hold a sensor.

In some variations, the techniques described herein relate to a system, wherein the syringe includes a finger rest including a housing configured to hold a sensor.

In some variations, the techniques described herein relate to a system, wherein the three-dimensional image includes an optical face mesh.

In some variations, the techniques described herein relate to a system, wherein the tracker is electromagnetic.

In some variations, the techniques described herein relate to a system, wherein the tracker is optical.

In some variations, the techniques described herein relate to a system, wherein the tracker is LIDAR-based.

In some variations, the techniques described herein relate to a system, further including a wearable headband configured for use on the patient as a stable reference frame for motion compensation, wherein the wearable headband includes a cradle configured to receive the tracker.

In some variations, the techniques described herein relate to a system, wherein the cradle includes a retention mechanism and an anti-rotation feature.

In some variations, the techniques described herein relate to a system, wherein the wearable headband is configured to be integrated with optical sensors or electromagnetic sensors.

In some variations, the techniques described herein relate to a system, further including a mount coupled to the ultrasound probe.

In some variations, the techniques described herein relate to a system, wherein the mount includes a first interface configured to couple with the ultrasound probe and a second interface configured to couple with the tracker.

In some variations, the techniques described herein relate to a system, wherein the mount is configured to provide kinematic constraint of six degrees of freedom.

In some variations, the techniques described herein relate to a system, wherein the mount is configured to provide repeatable and stable reattachment without recalibration of the ultrasound probe.

In some variations, the techniques described herein relate to a method for planning an aesthetic procedure on a patient, the method including: generating a three-dimensional image of a face of the patient using a tracker, wherein the three-dimensional image includes an optical face mesh; generating an ultrasound image of the face of the patient using an ultrasound probe, wherein the ultrasound image includes ultrasound-derived volumetric data; registering the optical face mesh and the ultrasound-derived volumetric data into a patient-specific composite model; identifying anatomical features using the patient-specific composite model; generating a risk map including one or more safety regions and one or more risk regions based on spatial proximity of the vasculature of the patient to calculated injection trajectories; evaluating the patient-specific composite model and the risk map to select one or more target locations defined by the risk map; and digitally presenting the one or more target locations for treatment directly upon the face of the patient.

In some variations, the techniques described herein relate to a method, further including treating the one or more target locations based on the one or more target locations presented upon the face of the patient.

In some variations, the techniques described herein relate to a method, wherein treating includes injecting the one or more target locations with a filler material via a syringe, wherein the syringe is guided by analysis of the composite model.

In some variations, the techniques described herein relate to a method, wherein the ultrasound probe is coupled to the tracker.

In some variations, the techniques described herein relate to a method, further including creating a simulated model based on a volumetric effect of injections at the one or more target locations.

In some variations, the techniques described herein relate to a method, wherein creating the simulated model includes selecting the one or more target locations with a stylet.

In some variations, the techniques described herein relate to a method, wherein the one or more target locations is associated with one or more parameters selected from: injection trajectory, target depth, and injection volume.

In some variations, the techniques described herein relate to a method, further including an augmented reality device configured to be worn by a practitioner to provide live visualization of the composite model and the patient during injection, wherein the augmented reality device overlays the composite model over the patient.

In some variations, the techniques described herein relate to a method, wherein evaluating the composite model further includes evaluating a simulated model of predicted volumetric effects to determine injection location, injection trajectory, and injection volume.

In some variations, the techniques described herein relate to a method, further including: tracking the face of the patient in real time; inferring facial expression using a classification algorithm; and dynamically deforming the patient-specific composite model to compensate for expression-induced tissue displacement during injection.

### BRIEF DESCRIPTION OF THE DRAWINGS

FIG. 1 illustrates a method for performing imaging of a patient's face and subsequently performing an aesthetic procedure in accordance with one variation of the present invention.

FIG. 2 illustrates an illustration of various steps of the method in accordance with one variation of the present invention.

FIG. 3 illustrates an augmented reality system for use in accordance with one variation of the present invention.

FIGS. 4A to 4F illustrate various views of the practitioner comprising the treatment plan and the patient during the procedure.

FIG. 5 illustrates a method for preparing a treatment plan in accordance with one variation of the present invention.

FIG. 6 illustrates a method for preparing a segmentation mask from ultrasound data in accordance with one variation of the present invention.

FIG. 7 illustrates a method for preparing a 3D composite model in accordance with one variation of the present invention.

FIG. 8 illustrates a method for preparing a 3D composite model and a heat map of the patient's anatomy in accordance with one variation of the present invention.

FIG. 9 illustrates an architecture diagram of the system in accordance with one variation of the present invention.

FIG. 10 illustrates a headband with a detachable sensor in accordance with one variation of the present invention.

FIG. 11 illustrates an ultrasound probe with an electromagnetic tracking sensor in accordance with one variation of the present invention.

FIG. 12 illustrates a variation of a syringe having a sensor embedded within in accordance with one variation of the present invention.

### DETAILED DESCRIPTION OF THE INVENTION

Systems and methods disclosed herein can be integrated systems that use augmented reality (AR), ultrasound imaging, localization systems, and planning algorithms to assist in facial aesthetic procedures. The system can enable precise 3D modeling of facial anatomy, planning of injectable treatments, and real-time guidance during procedures. Applications thereof can include facial injectables, education, and laser-based treatments.

The system can create a detailed 3D imagery of the patient's anatomy and vasculature and subsequently develop and simulate patient-specific treatment while avoiding vasculature, resulting in safely injecting material with precise real-time image guidance.

As will be discussed further herein, the system can comprise an integrated tracking system to localize the ultrasound probe, face, and objects in a shared coordinate frame, software algorithms to reconstruct 3D anatomical models from a series of localized ultrasound images, planning software to generate patient-specific injection plans, and an augmented reality display system to project anatomical overlays and procedure guidance.

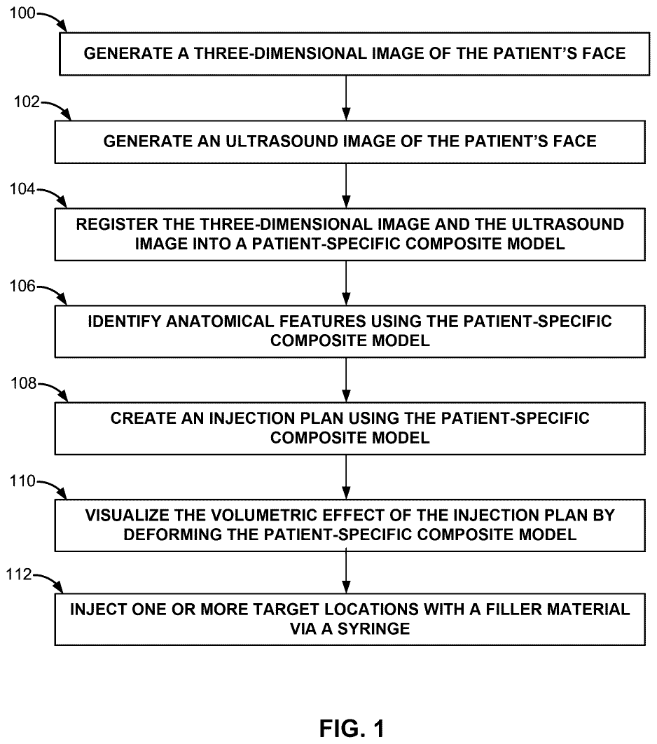

As seen in FIG. 1, a method of performing an aesthetic procedure can first comprise the step 100 of acquiring patient surface and depth data by acquiring a three-dimensional optical representation of a patient's face. To facilitate this, a bi-modal localization device (e.g., an electromagnetic and/or optical sensor) can be temporarily adhered to the patient's facial skin at a position spaced from the anatomical region identified to be scanned, thereby minimizing occlusion and ensuring stable tracking during data acquisition. The practitioner can then capture a series of color and depth images using a stereo or LIDAR-based optical sensor integrated into a mobile computing platform (e.g., via a laptop, smartphone, or a tablet, etc.). The collected image set can form the raw surface and depth dataset used for subsequent 3D reconstruction and registration operations.

Using the captured images, the processor can reconstruct a high-resolution three-dimensional (3D) mesh of the patient's facial surface using photogrammetric reconstruction techniques (e.g., multi-view stereo, structure-from-motion) or structured-light reconstruction algorithms. The reconstructed 3D model can then be rectified and spatially normalized to a standardized coordinate frame, for example, by using pose-estimation data from an external or integrated localization tracker to correct for variations in camera orientation, patient motion, or lens distortion.

The processor can further execute one or more trained convolutional neural networks (CNNs) configured to automatically detect and segment facial landmarks (e.g., medial and lateral canthi, nasion, alar bases, oral commissures, etc.). In some variations, the processor can identify contour features such as curvature extrema or surface normals. The refined and annotated optical data can be stored in memory as a registered 3D facial mesh model that digitally represents the patient's surface anatomy.

Step 102 comprises the generation of patient ultrasound data via an ultrasound probe. The ultrasound probe can be used to scan regions of the patient's face to capture subsurface anatomical information, including soft-tissue layers, vasculature, and skeletal contours. The ultrasound probe can include or can be coupled to a localization device (e.g., an electromagnetic or optical tracker) configured to provide real-time positional and orientational data during image acquisition.

The ultrasound probe can further comprise tracking markers that capture a series of localized 2D images, which are combined using software algorithms to generate a 3D model of the patient's facial anatomy. In some variations, the tracking markers can comprise integrated fiducial markers built into the housing of the ultrasound probe.

A series of spatially indexed 2D ultrasound images is captured as the practitioner sweeps the probe across the target region. The images can then be computationally combined using probe-tracking data and volumetric reconstruction algorithms to generate a registered 3D ultrasound model of the patient's facial anatomy.

The ultrasound probe can be handheld in some variations, though the ultrasound probe does not necessarily need to be used during the injection procedure.

In some variations, the optical tracker can comprise a headset with one or more stereo cameras configured to localize the probe, face, hands, and syringe.

During scanning, B-mode ultrasound frames can be time-synchronized with the spatial pose of the ultrasound probe and the patient to generate a spatially coherent dataset suitable for volumetric reconstruction. Calibration between the image coordinate frame of the probe and its tracking frame can be performed using a predefined calibration method such as a phantom-based or automatic feature-based calibration technique. The collected data can be cleaned and/or filtered to remove noise and errant frames and converted into the same coordinate reference as the patient optical model of step 100.

In some variations, the system can achieve minimum tracking tolerances of approximately 0.25 mm (i.e., the position of the tracked object can be detected with a maximum deviation of 0.25 mm from its actual location).

In some variations, the calibration precision of the system can be around 1 mm (i.e., when the system is calibrated, the reference alignment or baseline measurement is accurate to within 1 mm.)

Step 104 comprises the registration of the optical data and the ultrasound data gathered from steps 100 and 102. The optical facial mesh and the ultrasound-derived volumetric data can be registered into a single, patient-specific anatomical model. Corresponding anatomical features (e.g., bony landmarks, vessel bifurcations, dermal-subdermal interfaces, or identifiable curvature extrema, etc.) can be identified either manually by the practitioner via a tracked pointer or automatically using landmark recognition systems executed by the processor.

The system can then compute a spatial transformation between the ultrasound and optical coordinate frames using an iterative closest point (ICP) or a coherent point drift (CPD) algorithm. The processor can then generate a composite, deformable 3D model that has identified anatomical features and integrates both the external surface mesh and the underlying subsurface anatomical structures, providing a fused or composite model of the patient's facial anatomy for subsequent visualization, planning, or guidance operations.

In some variations, the composite model can be deformed using physics-based mesh registration to correct for patient motion or tissue elasticity.

Step 106 comprises anatomical segmentation and risk mapping of the model in order to evaluate and analyze the composite model for treatment. In this step, the fused composite model can be processed to identify relevant anatomical layers and vascular structures for potential injection sites. To this end, a trained segmentation model (e.g., 3D U-Net) can classify anatomical regions corresponding to vessels, muscles, fat pads, and bone. From the segmented data, the system can generate a “go” and “no-go” heatmaps based on the spatial proximity of injection paths to sensitive structures such as arteries or nerves. These safety maps can be stored as overlay layers for both visualization during subsequent procedural steps and subsequent planning steps.

Accordingly, the method step can generate a risk map comprising one or more safety regions and one or more risk regions based on spatial proximity of the vasculature of the patient to calculated injection trajectories.

Step 108 comprises the creation of a treatment plan for predictive planning and counseling before injection. In this step, the practitioner can interact with the patient-specific 3D model through a user interface that allows visualization, manipulation, and modification of proposed treatment parameters. The practitioner can use a tracked pointer or stylus to select one or more target locations for injection sites on the patient's face corresponding to the 3D facial model. Each injection site can be associated with parameters including, but not limited to: injection trajectory, target depth, and injection volume. The user interface can further allow adjustment of these parameters based on real-time feedback from the risk maps and anatomical segmentations previously generated. The finalized treatment plan can then be stored as a procedural blueprint for subsequent execution, visualization, or intra-procedural guidance.

The treatment plan can comprise at least one set of: one or more needle insertion points on the patient's anatomy, one or more target locations within the patient's anatomy, and a dosage of material to be deposited. Accordingly, treatment guidance can comprise the visualization of the syringe relative to the patient anatomy. The treatment guidance can display simulated ultrasound images based on the syringe's relative pose to patient anatomy, emulating the ultrasound device.

In some variations, the treatment plan can be generated by modifying templates that encode standard injection patterns for aesthetic procedures.

In some variations, the treatment plan can be used solely for generating a template for future procedures and analysis.

In some variations, the system can record one or more parameters in relation to the patient's coordinate frame and can allow for editing via a graphical user interface or voice command. The resulting dataset constitutes a preoperative treatment plan and image/model.

Step 110 comprises creating a simulation of the predicted outcome of the injection procedure in order to visualize the volumetric effect of the injection plan by deforming the composite model. The simulation engine can deform the patient-specific model to generate a visual representation of the volumetric effect of the proposed injections. The patient-specific composite model can be dynamically deformed to compensate for any expression-induced tissue displacement during injection. The engine can apply a physics-based solver such as a finite element model (FEM) or position-based dynamics (PBD) to approximate tissue displacement and filler distribution. The simulated model can be rendered for pre-procedure visualization, visualizing anticipated volumetric augmentation, contour adjustments, and surface profile changes. The simulation data can also be used for patient counseling, training, or preoperative comparison.

Step 112 comprises performing the procedure with real-time guidance. During the procedure, the system can provide real-time image guidance to the practitioner to assist the practitioner in navigating the planned injection paths. The syringe can be tracked by a markerless computer-vision algorithm or by a tracking marker (electromagnetic or optical) coupled to the syringe for accurate treating of the patient's face. Simultaneously, an optical markerless localization system can track a patient's face in real-time, compensating for patient movement, breathing, or subtle posture changes, while a classification algorithm can infer the patient's facial expression. Based on the detected expression of the patient, the patient's anatomical model can be morphed to match, allowing compatibility with clinical workflows where different facial expressions are emoted for specific targeted injections. This real-time synchronization between the physical patient, the tracked syringe, and the updated 3D anatomical model enables accurate guidance, safety awareness, and adherence to the planned injection strategy.

In some variations, the system can use object recognition and hand-tracking algorithms to determine the position and orientation of the syringe during the procedure, eliminating the need for physical markers.

The processor can continuously determine the syringe tip position and orientation relative to the patient model and can display a simulated ultrasound slice aligned with the actual injection trajectory. The guidance interface can warn the user if the planned trajectory intersects any restricted (“no-go”) anatomical regions identified in step 106.

In some variations, an augmented reality headset can provide live visualization of the guidance information. The headset camera can identify patient facial landmarks and can align the virtual patient model to the patient using visual-inertial odometry. The headset can overlay segmented anatomy, injection trajectories, and safety heatmaps directly onto the patient's face within the practitioner's field of view. During the procedure, the augmented reality overlay can update dynamically in real-time to reflect patient or instrument movement, while maintaining registration within predefined tolerance limits. This enables consistent alignment with the patient-specific anatomical model and the planned procedural pathway, supporting accurate, heads-up guidance throughout the intervention.

In some variations, the system can periodically recalibrate the ultrasound probe or tracking system using an automatic phantom-based procedure to maintain accuracy.

In some variations, the system can store all optical, ultrasound, and tracking data in encrypted format for later review, comparison, or integration into longitudinal patient records.

In some variations, post-procedure data can be anonymized and uploaded to a remote server for model retraining to improve segmentation or outcome prediction accuracy.

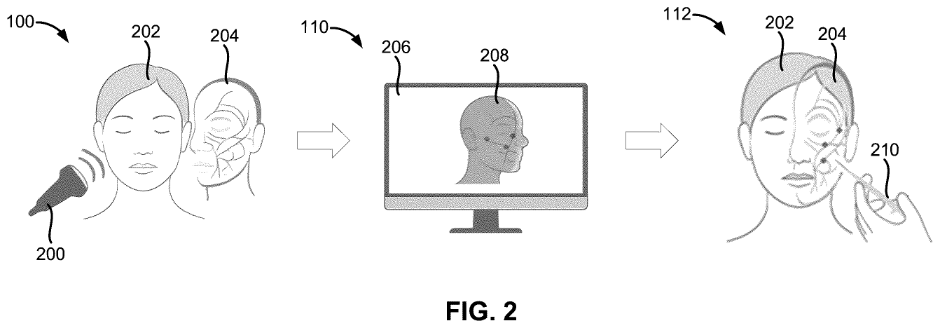

FIG. 2 illustrates various steps of the method of FIG. 1. Step 100 comprises employing a bi-modal localization device, such as an electromagnetic (EM) tracker and/or an optical tracking sensor 200 positioned on the patient 202 to acquire high-fidelity, patient-specific 3D model 204 of anatomical structures and vasculature. The localization device allows real-time spatial tracking and provides precise registration between the patient and the imaging system, enabling accurate reconstruction of vascular pathways, soft tissue contours, and other relevant anatomical landmarks. It should be understood that the electromagnetic (EM) tracker and the optical tracking sensor can be used either alone or in combination.

In step 110, a display 206 presents the patient-specific treatment plan 208, highlighting critical structures to avoid, such as major blood vessels or nerves. The visualization can include volumetric overlays, color-coded safety zones, or interactive guidance cues to enhance practitioner situational awareness during planning and execution.

Step 112 comprises performing the procedure on the patient 202 using the acquired 3D model 204 for guidance. The clinician administers injections via a syringe 210. The system ensures that the syringe tip trajectory is dynamically displayed, enabling precise delivery of filler material while minimizing risk to surrounding vasculature and soft tissue.

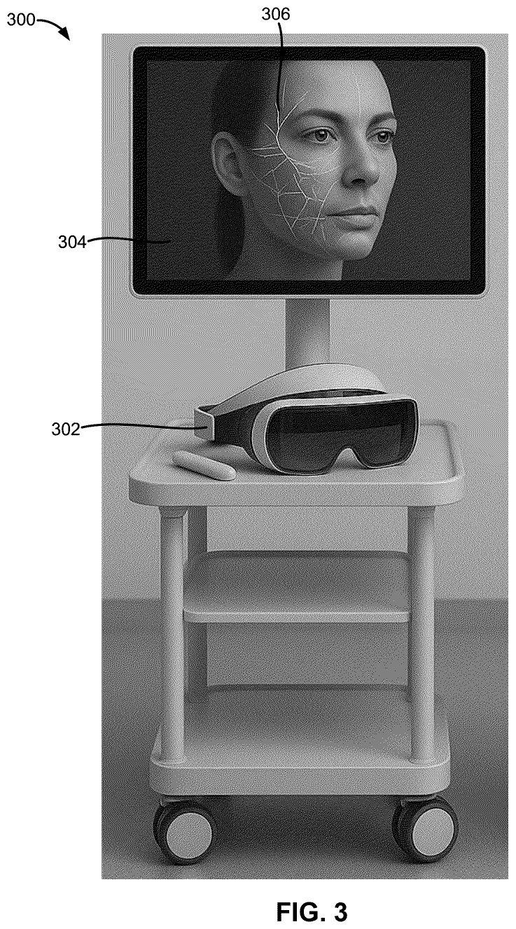

FIG. 3 illustrates an example of an augmented reality system 300 for use with step 112, performing the procedure on the patient. The augmented reality system 300 can comprise an AR headset 302 worn by the practitioner, enabling direct visualization of the patient-specific composite model overlaid on the real patient in real time. The headset 302 can identify key facial landmarks and register the patient-specific composite model to the patient's anatomy using visual-inertial odometry, ensuring accurate spatial alignment even with subtle head movements.

The augmented reality system 300 can overlay segmented anatomical structures, planned injection trajectories, and safety heatmaps directly onto the patient's face, providing continuous guidance during the procedure. The overlays can dynamically update in real time to reflect patient motion or instrument movement.

The augmented reality system 300 can also include a display 304 that presents the patient-specific model 306, providing the practitioner with an additional visual reference. The display 304 can serve as a real-time procedural monitor, showing guidance information, anatomical overlays, and safety indicators.

In some variations, the practitioner can forgo the augmented reality system 300 in favor of performing the procedure with the display. In some variations, the injection procedure can be performed using robotic assistance.

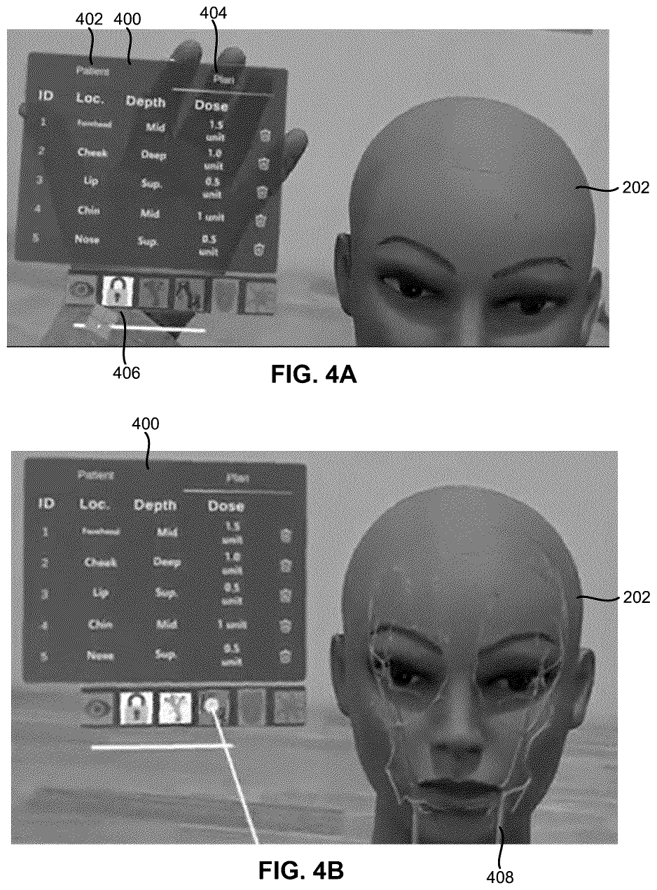

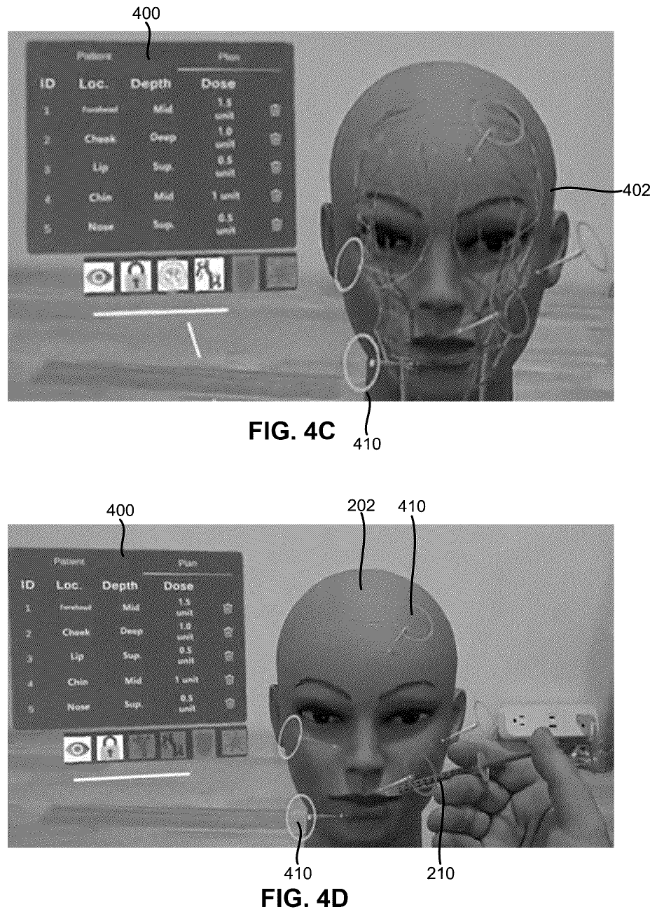

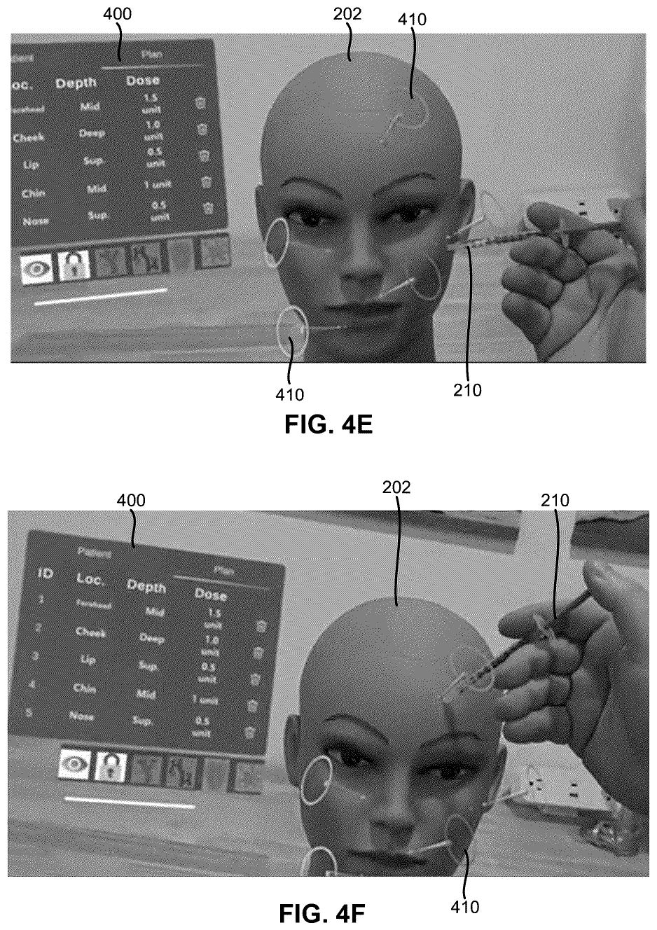

FIGS. 4A to 4F illustrate various views of the practitioner comprising a treatment plan 400 presentinga menu or summary which can be actuated to appear permanently or temporarily within the visual field of the headset 302 worn by the practitioner and/or upon the display 304 during the procedure. The patient 202 is also shown during the procedure. The treatment plan 400 can comprise a “Patient” tab 402 and a “Plan” tab 404. The “Patient” tab 402 can be used by the practitioner to select a specific patient and/or the corresponding procedural plan based on the current treatment session. The “Plan” tab 404 can comprise various parameters for performing the treatment plan, such as an ID number, a location on the patient's face, a depth of injection, and the planned dose of filler material to be delivered.

A menu 406 of the treatment plan can comprise various modes of visualization for the practitioner. As seen in FIG. 4B, for example, the practitioner can view the modeled vasculature 408 of the patient according to the patient-specific composite model. This allows the practitioner to overlay the modeled vasculature 408 over patient 202 during the procedure.

As seen in FIG. 4C, the treatment plan 400 can further define one or more injection points 410 for a particular procedure. The one or more injection points 410 can be represented by a circle of entry and a line depicting an angle of trajectory of a syringe or instrument to be held relative to the patient's face. Once the one or more injection points are visualized by the practitioner, the practitioner has the option to remove the modeled vasculature 408, as seen in FIGS. 4D to 4F, providing for a clearer overlay during the procedure.

During injection of, e.g., the filler material, into the patient's face via syringe 210, the practitioner can rely on the treatment plan 400 and the one or more injection points 410 to guide precise needle placement, angle, and depth, enabling safe and accurate injections.

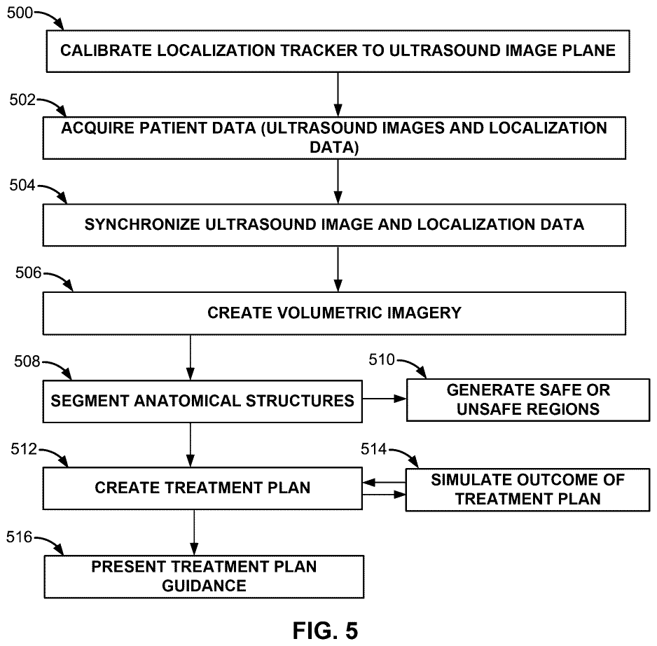

FIG. 5 illustrates a method for preparing a treatment plan in accordance with one variation of the present invention. Step 500 comprises calibrating the localization tracker (or EM/optical sensor) to the ultrasound image plane. Step 502 comprises acquiring patient-specific data as described in steps 100 and 102 above, which can include tracked ultrasound sweeps, surface scans, or additional imaging modalities. In step 504, the various data streams collected in step 502 are temporally and spatially synchronized. Step 506 comprises generating a reconstructed volumetric dataset based on the synchronized inputs, after which the system segments the relevant anatomical structures in step 508.

Using the segmented anatomical structures, the system identifies and generates treatment-relevant safety regions, including safe (“go”) zones and unsafe (“no-go”) zones, in step 510. In step 512, a treatment plan is generated (in conjunction with step 108 above) based on these zones, the patient's anatomy, and procedural parameters. Step 514 comprises simulation of the proposed treatment plan, enabling the practitioner to visualize injection paths, assess potential risks, or evaluate target structures. The practitioner reviews the simulation results and, in step 516, determines whether the treatment plan is suitable for presentation to the patient.

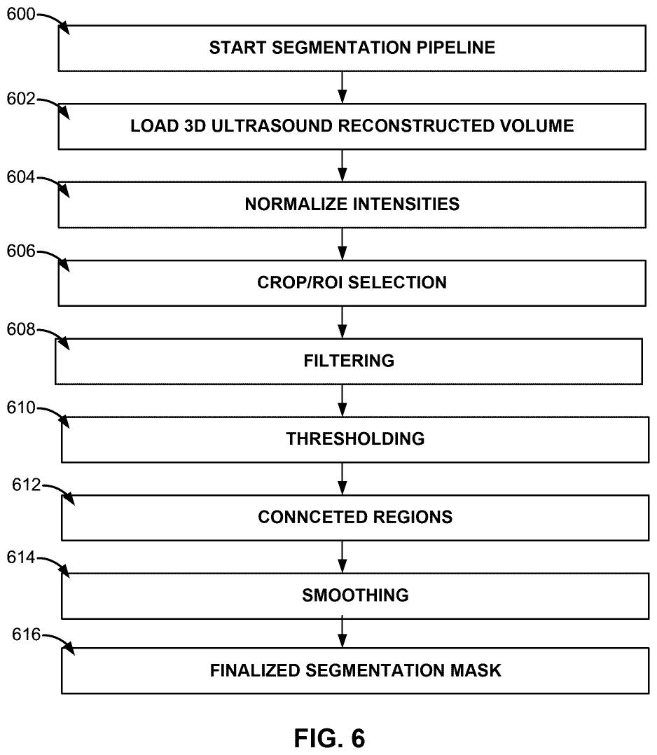

FIG. 6 illustrates a method for preparing a segmentation mask from ultrasound data in accordance with one variation of the present invention. The segmentation pipeline can begin at step 600, which may incorporate or correspond to initialization step 106 described above. In step 602, the system loads the reconstructed 3D ultrasound volume generated from freehand or tracked-acquisition data. Steps 604, 606, and 608 collectively implement a pre-processing stage. Step 604 can comprise normalizing intensities to a standardized dynamic range to reduce variance. Step 606 can comprise selecting a region of interest (ROI) or applying a cropping operation to focus subsequent processing on anatomical structures of relevance. Step 608 can comprise applying additional filtering operations, such as median filtering, Gaussian smoothing, or speckle-reducing filters.

Steps 610, 612, and 614 implement a post-processing stage following inference by a neural-network-based segmentation model. Step 610 can comprise applying an intensity or probability threshold to convert the model output into a binary or probabilistic mask. Step 612 can comprise performing connected-region analysis to remove small isolated regions. Step 614 can comprise optional smoothing operations, such as morphological closing, surface smoothing, or volumetric dilation/erosion, to refine boundaries and reduce artifacts. The finalized segmentation mask is produced in step 616 and can be subsequently used for visualization, measurement, navigation, or a combination thereof.

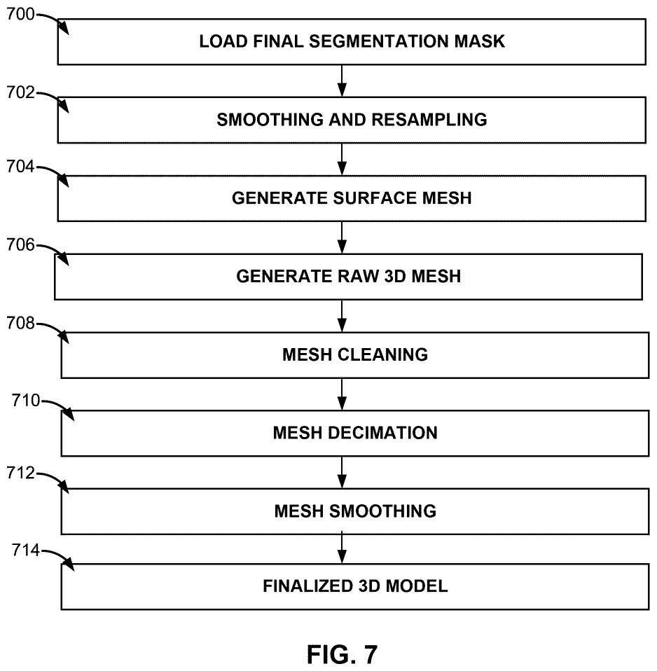

FIG. 7 illustrates a method for preparing a 3D composite model in accordance with one variation of the present invention. The 3D modeling pipeline begins at step 700, which loads the finalized segmentation mask produced in step 616 above. In step 702, the system performs a pre-processing stage on the segmentation mask, which can include smoothing operations (e.g., Gaussian or morphological smoothing) and spatial resampling for mesh extraction. Steps 704 and 706 respectively comprise generating an initial surface mesh using a marching-cubes algorithm and producing a corresponding raw 3D mesh consisting of vertices and faces.

Steps 708, 710, and 712 together form a post-processing stage applied to the raw mesh. Step 708 comprises mesh cleaning, which can involve the removal of non-manifold elements, the elimination of small artifacts, and the enforcement of surface continuity. Step 710 comprises mesh decimation to reduce triangle count. Step 712 comprises mesh smoothing to improve surface quality and reduce high-frequency irregularities. In step 714, the system outputs the finalized 3D composite model, which can then be used for visualization, registration, treatment planning, or integration with augmented-reality guidance systems.

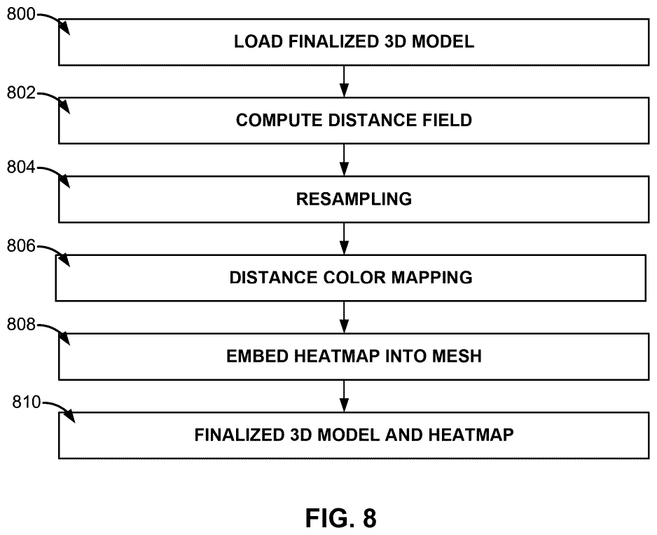

FIG. 8 illustrates a method for preparing a 3D composite model and a heat map of the patient's anatomy in accordance with one variation of the present invention. The pipeline begins at step 800, which loads the finalized 3D composite model generated in step 714 above. In step 802, the system computes a distance field (e.g., signed distance field or Euclidean distance field) relative to one or more anatomical surfaces or segmented structures of interest. Step 804 comprises optionally resampling the distance field to achieve consistent spatial resolution or to optimize downstream visualization performance. In step 806, the system applies distance-to-color mapping to convert the computed distance values into scalar heatmap values. Step 808 comprises embedding the heatmap data into the 3D mesh by assigning vertex colors or generating a texture map that encodes the heat values. Step 810 outputs the final 3D composite model together with its associated heatmap.

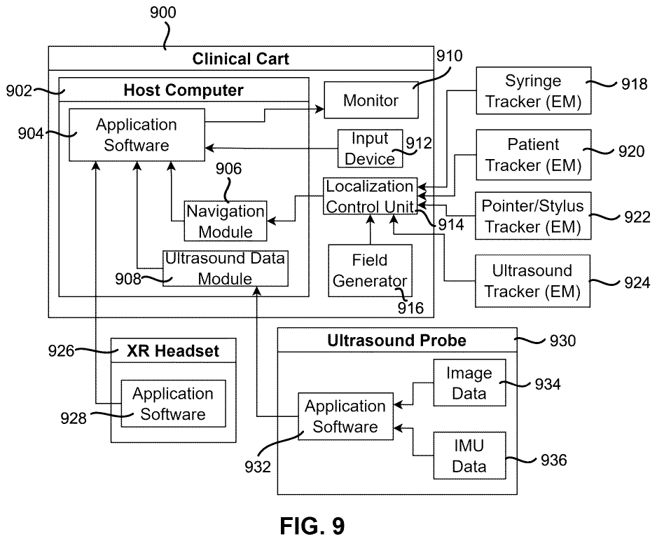

FIG. 9 illustrates an architecture diagram of the system in accordance with one variation of the present invention. A clinical cart 900 can comprise a host computer 902 comprising the application software 904 for the system, a navigation module 906, and an ultrasound data module 908. The host computer 902 can be coupled to a monitor 910 and an input device 912. A localization control unit 914 can be coupled to the navigation module 906, while a field generator 916 is coupled to the control unit 914.

A variety of electromagnetic components can be coupled to the localization control unit 914, such as a syringe tracker 918, a patient tracker 920, a pointer/stylus tracker 922, and an ultrasound tracker 924.

The application software 904 can be coupled to an XR headset 926, which includes its own application software 928. The ultrasound data module 908 can be coupled to an ultrasound probe 930, which can comprise an application software 932, which can hold image data 934 and IMU data 936.

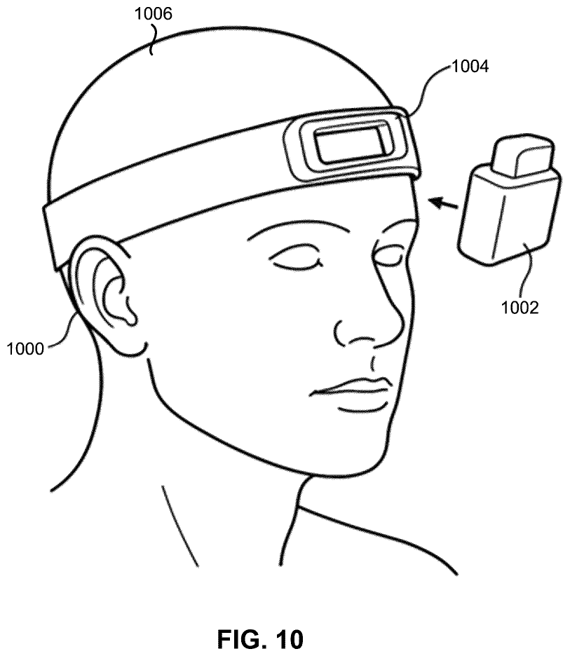

FIG. 10 illustrates a wearable headband 1000 incorporating a non-disposable electromagnetic (EM) tracking sensor 1002 positioned on a cradle 1004. The headband 1000 can provide a stable, unobtrusive reference frame for monitoring patient head 1006 and face motion during facial aesthetic procedures, including ultrasound scanning, AR-based or software-based planning, and image-guided injections. In contrast to adhesive facial patches, which can obstruct the treatment field or distort with facial movement, the headband-mounted sensor 1002 can remain rigidly coupled to the cranium, maintaining consistent spatial registration relative to the skull. The headband 1000 can be configured to avoid contact with target injection sites, minimizing interference with the procedure and eliminating the need for repeated calibration during use. Accordingly, the headband 1000 can remain in place throughout the entirety of the procedure, providing continuous motion tracking while preserving unobstructed visibility and compatibility with established injection workflows.

In some variations, the headband 1000 may be made from textile, silicone, elastomer, polymer composites, or adjustable straps with Velcro or buckle-style fasteners, or any combination thereof.

The headband 1000 can comprise a flexible or semi-rigid circumferential band sized to fit around the patient's head 1006. The headband 1000 can comprise a front, side, or temporal mounting location optimized for minimal skin shift. The headband can comprise an integrated sensor docking cradle 1004 that receives the non-disposable EM tracking sensor 1002.

The built-in cradle 1004 can create a repeatable, non-destructive mechanical interface between the headband 1000 and the reusable EM sensor 1002. The cradle 1004 can comprise a deterministic positioning geometry (e.g., cone-vee-flat, sphere-seat, or keyed rails).

The cradle 1004 can further comprise a retention mechanism such as: a magnetic preload, spring detents, snap-fit tabs, a quarter-turn bayonet, or a combination thereof.

The cradle 1004 can comprise anti-rotation features ensuring that the mounted sensor 1002 always assumes the same orientation relative to the headband 1000. Because the cradle 1004 is built into the headband, and the EM sensor 1002 is not bonded to the cradle 1004, the EM sensor 1002 can be inserted and removed without wear, adhesive residue, or replacement of components.

The headband 1000 can be positioned around the patient's head such that the sensor cradle aligns with bony, low-deformation regions (e.g., the frontal bone, the upper lateral orbit, the parietal region). The headband 1000 can apply consistent circumferential tension to maintain rigid coupling to the skull, reducing artifacts from facial skin movement (e.g., expressions, smiling, speech).

The headband 1000 can be placed on hair-covered regions since the headband does not rely on adhesive contact and retains stability through mechanical compression. This design ensures that the sensor 1002 tracks gross cranial motion with high accuracy while leaving the entire face unobstructed for dermal filler injections, neurotoxin injections, ultrasound scanning, AR visualization, laser and energy-based treatments, or a combination thereof.

In some variations, no pivot or geometric calibration is needed as the sensor's role is to define a stable reference frame for patient head motion. Any absolute spatial offset between the EM sensor 1002 and the patient's facial anatomy can be accounted for through software-based registration to ultrasound volumes, 3D facial meshes, or other imaging inputs obtained during pre-procedural scanning. Once the EM sensor 1002 is secured within the headband cradle, real-time tracking of head position and orientation can commence immediately, enabling continuous motion compensation throughout subsequent imaging, planning, and injection steps without interrupting the clinical workflow.

In some variations, the sensor 1002 can enable real-time motion compensation for the patient's head during ultrasound volume acquisition. The sensor 1002 can further serve as a stable anchor point for AR overlays, vascular maps, and injection plans, and a consistent reference frame for coordinating a tracked syringe, tracked probe, or robotic assistance. The headband thus can provide the system with hands-free operation that does not interfere with injector ergonomics or access to treatment zones.

One technical problem encountered by the inventors is securing a localization sensor to the patient's head or face rigidly and repeatably, without interfering with the aesthetic treatment area. For example, the use of adhesive facial patches can block injection zones. One technical solution discovered by the inventors is to use the headband to anchor the sensor to the skull via circumferential tension, in order to attach the localization sensor to the patient such that facial motion is faithfully captured. The headband can accordingly comprise a mechanical dock (e.g., kinematic mount) for a reusable EM or optical sensor.

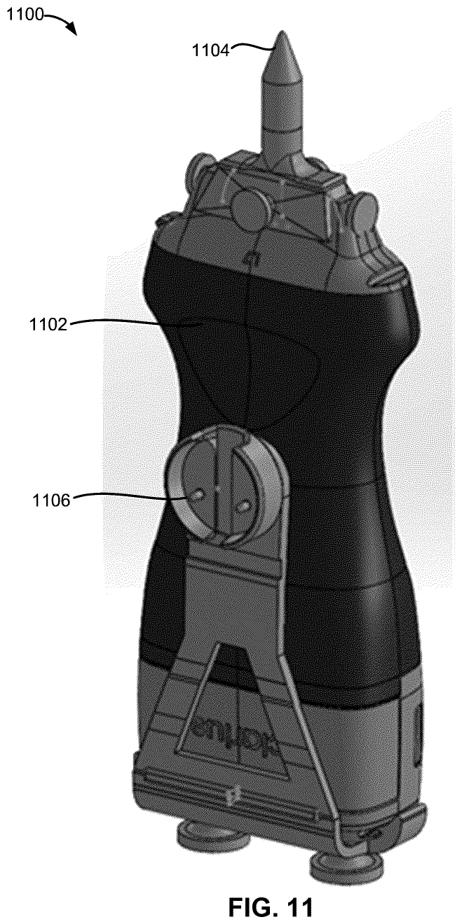

FIG. 11 illustrates a system 1100 comprising hardware components and associated calibration methods that enable repeatable and precise alignment of a localization sensor (e.g., an EM tracking sensor) to a tracked ultrasound probe 1102. The system can be configured to maintain a known and stable spatial relationship between the sensor and the ultrasound image plane, ensuring that positional accuracy is preserved after multiple detachments, reattachments, or adjustments of the sensor relative to the probe. This stable alignment allows the processor to consistently map acquired ultrasound frames into a global reference frame, facilitating reliable registration with optical facial models.

In some variations, the system 1100 can comprise a secondary calibration attachment that temporarily interfaces with the probe to perform a pivot-based tip 1104 calibration, which is then used to compute the spatial offset between the EM sensor and the ultrasound image plane. This offset can become the basis for accurately mapping each 2D ultrasound frame into 3D space and enabling real-time 3D ultrasound volume creation.

The probe 1102 can be used to enable repeatable positioning of the EM sensor on the ultrasound probe with minimal positional variance (about ≤2 mm in translation and about ≤2 degrees in rotation), allowing reliable calibration across procedures.

The ultrasound probe 1102 can comprise a kinematic mount 1106 which can comprise a probe-side interface, a sensor-side dock, and a three-point deterministic positioning geometry. The probe-side interface can be affixed to or integrated within the ultrasound probe housing. The sensor-side dock can hold the EM sensor (not shown). The three-point deterministic positioning geometry can be a Maxwell kinematic mount 1106, for example, a cone, a vee-groove, and a flat or sphere-seat. The kinematic mount 1106 can also comprise mechanical capture features such as a spring preload or magnet preload and/or a mechanical latch or snap-fit.

The kinematic mount 1106 can constrain all six degrees of freedom and can ensure that when the sensor module is removed and reattached, the offset transformation between the EM sensor frame and the probe physical coordinate frame remains constant. Additionally, no recalibration is required unless the mechanical interface is replaced or damaged, enabling fast clinical workflows and consistent tracking accuracy.

In some variations, a removable calibration attachment, or a tip 1104 can connect to the distal end (imaging end) of the ultrasound probe. The calibration attachment can comprise a rigid extension projecting outward from the probe body and a precision-machined tip (e.g., a stylet or spherical probe) positioned at a known fixed transform relative to the ultrasound transducer and its image plane. The attachment can further comprise a removable or snap-on interface designed to seat in a repeatable orientation relative to the probe housing or a keyed docking feature, ensuring consistent alignment across installations.

In some variations, a small calibration fixture containing a divot or hemispherical recess can be placed on a stable platform on the probe 1102. In this variation, the probe 1102, with the calibration attachment installed, can be positioned such that the tip of the attachment sits within the fixture. The practitioner can pivot, rotate, or otherwise manipulate the probe about the fixed tip location while the EM sensor records its six-degree-of-freedom pose over a series of frames. A pivot-based calibration algorithm can then compute the precise position of the tip relative to the EM sensor and derive the transformation matrix from the sensor's coordinate system to the probe's transducer tip. This transformation can subsequently be used to accurately map each acquired 2D ultrasound frame into 3D space, enabling precise volumetric reconstruction and integration with patient-specific models.

Once the tip offset is known, the known geometric relationship between the calibration tip and the ultrasound transducer face (or central ray of the ultrasound beam) allows calculation of the ultrasound image-plane origin relative to the EM sensor. The system can then apply this transformation to map each ultrasound pixel or voxel coordinate into a global 3D reference frame, enabling accurate freehand 3D ultrasound volume reconstruction, real-time tracking of anatomical structures, and integration with augmented reality-guided injection systems or robotic-assisted procedural platforms.

By establishing this calibrated spatial correspondence, the system can ensure that all acquired ultrasound frames are consistently registered in space, supporting downstream applications such as 3D elastography, vascular mapping, needle navigation, and other procedures.

Precise ultrasound probe tracking, combined with a repeatable mechanical mount for the EM sensor, can eliminate the need to repeat calibration every session. This can support consistent probe accuracy and an efficient workflow.

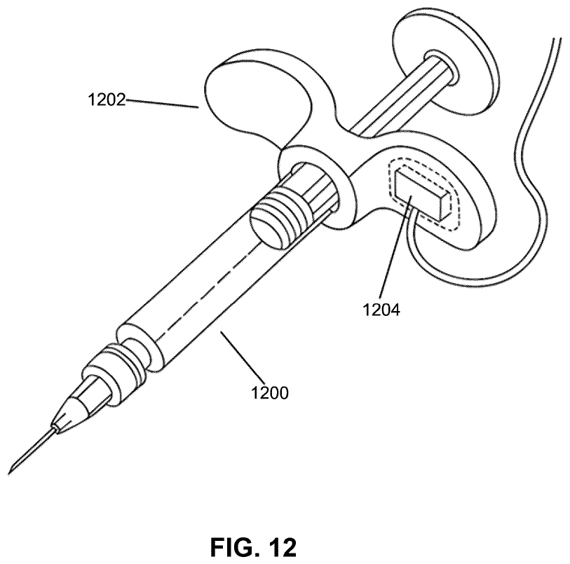

FIG. 12 illustrates a system for a localization sensor clip for a syringe 1200. The sensor clip 1202 can hold sensor 1204 and can be removable or integrated within the syringe 1200. The sensor clip system can enable precise spatial tracking of aesthetic or medical syringes using an embedded localization sensor 1204 (e.g., an electromagnetic (EM) tracking coil).

In some variations, the system can comprise a clip-on module containing the EM sensor 1204 that attaches rigidly to a standard syringe.

The system can further comprise a software workflow that allows the practitioner to specify the needle or cannula length, enabling accurate visualization and guidance during procedures such as dermal filler or toxin injections.

In some variations, the sensor clip 1202 can comprise a finger rest, and more specifically, a custom sensor-integrated finger rest.

In some variations, the system can comprise a method to calibrate the spatial offset from the EM sensor 1204 to the syringe tip using a pivot-based calibration.

The sensor 1204 can comprise a housing that contains the EM sensor coil. In some variations, the sensor can comprise on-board electronics for signal conditioning, amplification, or communication with a host processor.

The sensor 1204 can further comprise an attachment mechanism. In some variations, the attachment mechanism can be a flexible C-clip that removably snaps around the syringe barrel or finger rest. In some variations, the attachment mechanism can comprise a slotted collar that slides over the plunger cap. In some variations, the attachment mechanism can comprise a replaceable finger rest assembly with the sensor permanently embedded.

The sensor 1204 can further comprise an indexing geometry to ensure consistent orientation of the sensor relative to the syringe's long axis. The sensor can further comprise anti-rotation features, such as one or more of: internal flats, keyed surfaces, and dual-clip or tri-clip designs for three-point constraint.

In some variations, the sensor clip 1202 can be in close proximity to the finger rest such that the module clamps to the circular disk or finger support structure on the syringe 1200. The sensor clip can land on the rigid, repeatable geometry ensuring that each installation places the sensor at a predictable transform relative to the syringe barrel.

In some variations, the sensor clip 1202 can be located on the plunger cap of the syringe 1200. The module can attach around the plunger end, using a collar or snap-fit structure. This variation can be used for syringes without robust finger rests or when workflow for the practitioner necessitates attachment after loading the syringe.

In some variations, a custom finger rest can be provided on the syringe with the sensor embedded directly in the plastic mold to provide a rigid and consistent attachment.

The EM-sensor 1204 can be attached to the syringe and a calibration tip can be inserted into a calibration divot fixture, establishing a fixed point. In some variations, the calibration tip can correspond to the syringe tip. The syringe can be pivoted, rotated, and swept around the fixed point while the EM sensor 1204 records pose data. A standard pivot calibration algorithm can calculate the 3D position of the syringe tip relative to the EM sensor. The resulting rigid transformation, denoted as T (sensor→syringe tip), can represent the 3D translation and rotation that maps coordinates from the EM sensor's local coordinate frame to the syringe tip location. This transformation can be stored in association with the specific syringe type or sensor module, enabling accurate real-time mapping of the syringe tip during subsequent procedural guidance.

In some variations in which the sensor 1204 is integrated into the replacement finger rest, the system may not require an end-user pivot calibration. Because the geometry of aesthetic syringes is often standardized by brand and volume, the transformation between the embedded EM sensor and the syringe tip location can be factory-calibrated or predefined in software. Accordingly, when the practitioner selects the syringe type, brand, model, and volume from a user interface or database, the system can automatically retrieve a stored calibration transform mapping the EM sensor coordinates to the syringe tip (T (sensor→syringe tip)). This approach eliminates the need for manual pivoting or calibration procedures while maintaining accurate real-time positional tracking of the syringe tip during image-guided or augmented reality-assisted injections.

In some variations, the practitioner can select the needle length or cannula length from a list in the software. The system applies an additional transform offset to account for one or more of: length, gauge-dependent insertion depth, bevel angle, and cannula curvature. Different needle gauges can have different hub geometries, wall thicknesses, and effective tip offsets relative to the point where the needle connects to the syringe. Accordingly, the actual tip position is not the exact needle length measured from the hub, as the tip position can vary subtly with gauge.

The system can display the real-time 3D position and orientation of the syringe and needle tip, a virtual extension of the needle path, and a potential intersection of unsafe zones (e.g., vessels), treatment plans, or AR overlays to guide injections.

In some variations, the practitioner can use a 3D display device or a 2D monitor for real-time visualization.

One technical problem encountered by the inventors is rigidly attaching a localization sensor to a syringe so the system can track needle orientation and tip position. One technical solution discovered by the inventors is to provide a clip on finger-rest or plunger, or a replacement finger-rest with an embedded sensor to enable syringe tracking, necessary for navigation and guidance.

Such features may be used in any number of combinations with any of the variations described herein and such combinations are intended to be within the scope of this description.

It is apparent to one skilled in the art that various changes and modifications can be made to this disclosure, and equivalents employed, without departing from the spirit and scope of the invention. Elements shown with any variation are exemplary for the specific variation and can be used on or in combination with any other variation within this disclosure.

What is claimed is:

1. A system for planning an aesthetic procedure on a patient, the system comprising:
a tracker configured to generate a three-dimensional image of a face of the patient;
an ultrasound probe, the ultrasound probe configured to generate an ultrasound image of the face of the patient, wherein the ultrasound image and the three-dimensional image are registered into a patient-specific composite model; and
a display, wherein the display shows the patient-specific composite model created by identification of anatomical features of the patient-specific composite model, wherein the display shows a risk map derived from the patient-specific composite model, the risk map comprising one or more safety regions and one or more risk regions based on spatial proximity of the vasculature of the patient to calculated injection trajectories, wherein the patient-specific composite model is configured to be evaluated to select one or more target locations on the face of the patient constrained by the risk map.

2. The system of claim 1, further comprising a syringe configured to inject the one or more target locations with a filler material, wherein the syringe is guided by analysis of the patient-specific composite model.

3. The system of claim 2, wherein the syringe comprises a sensor module, the sensor module comprising a housing configured to hold a sensor.

4. The system of claim 2, wherein the syringe comprises a finger rest comprising a housing configured to hold a sensor.

5. The system of claim 1, wherein the three-dimensional image comprises an optical face mesh.

6. The system of claim 1, wherein the tracker is electromagnetic.

7. The system of claim 1, wherein the tracker is optical.

8. The system of claim 7, wherein the tracker is LIDAR-based.

9. The system of claim 1, further comprising a wearable headband configured for use on the patient as a stable reference frame for motion compensation, wherein the wearable headband comprises a cradle configured to receive the tracker.

10. The system of claim 9, wherein the cradle comprises a retention mechanism and an anti-rotation feature.

11. The system of claim 9, wherein the wearable headband is configured to be integrated with optical sensors or electromagnetic sensors.

12. The system of claim 1, further comprising a mount coupled to the ultrasound probe.

13. The system of claim 12, wherein the mount comprises a first interface configured to couple with the ultrasound probe and a second interface configured to couple with the tracker.

14. The system of claim 12, wherein the mount is configured to provide kinematic constraint of six degrees of freedom.

15. The system of claim 12, wherein the mount is configured to provide repeatable and stable reattachment without recalibration of the ultrasound probe.

16. A method for planning an aesthetic procedure on a patient, the method comprising:
generating a three-dimensional image of a face of the patient using a tracker, wherein the three-dimensional image comprises an optical face mesh;
generating an ultrasound image of the face of the patient using an ultrasound probe, wherein the ultrasound image comprises ultrasound-derived volumetric data;
registering the optical face mesh and the ultrasound-derived volumetric data into a patient-specific composite model;
identifying anatomical features using the patient-specific composite model;
generating a risk map comprising one or more safety regions and one or more risk regions based on spatial proximity of the vasculature of the patient to calculated injection trajectories;
evaluating the patient-specific composite model and the risk map to select one or more target locations defined by the risk map; and
digitally presenting the one or more target locations for treatment directly upon the face of the patient.

17. The method of claim 16, further comprising treating the one or more target locations based on the one or more target locations presented upon the face of the patient.

18. The method of claim 17, wherein treating comprises injecting the one or more target locations with a filler material via a syringe, wherein the syringe is guided by analysis of the composite model.

19. The method of claim 16, wherein the ultrasound probe is coupled to the tracker.

20. The method of claim 16, further comprising creating a simulated model based on a volumetric effect of injections at the one or more target locations.

21. The method of claim 20, wherein creating the simulated model comprises selecting the one or more target locations with a stylet.

22. The method of claim 20, wherein the one or more target locations is associated with one or more parameters selected from: injection trajectory, target depth, and injection volume.

23. The method of claim 16, further comprising an augmented reality device configured to be worn by a practitioner to provide live visualization of the composite model and the patient during injection, wherein the augmented reality device overlays the composite model over the patient.

24. The method of claim 16, wherein evaluating the composite model further comprises evaluating a simulated model of predicted volumetric effects to determine injection location, injection trajectory, and injection volume.

25. The method of claim 16, further comprising:
tracking the face of the patient in real time;
inferring facial expression using a classification algorithm; and
dynamically deforming the patient-specific composite model to compensate for expression-induced tissue displacement during injection.
